(19) United States
(12) Patent Application Publication
(10) Pub. No.: US 2026/0130862 A1
(43) Pub. Date: May 14, 2026

(54) ANTIMICROBIAL COMPOSITIONS AND METHODS OF USE THEREOF

(71) Applicants: University of Iowa Research Foundation, Iowa City, IA (US); National Jewish Health, Denver, CO (US)

(72) Inventors: Patrick M. Schlievert, Iowa City, IA (US); Donald YM Leung, Denver, CO (US)

(73) Assignee: University of Iowa Research Foundation, Iowa City, IA (US)

(21) Appl. No.: 19/361,433

(22) Filed: Oct. 17, 2025

Related U.S. Application Data
(60) Provisional application No. 63/708,337, filed on Oct. 17, 2024.

(51) Int. Cl. A61K 31/01; A61K 9/00; A61K 31/23; A61P 31/04

(52) U.S. Cl. CPC A61K 31/01; A61K 9/0014; A61K 31/23; A61P 31/04

(57) ABSTRACT

The invention provides antimicrobial composition comprising (a) a first active ingredient, wherein in the first active ingredient is selected from the group consisting of petroleum jelly, EpiCeram® emulsion, and/or glycerin, and (b) a second active ingredient selected from the group consisting of 1-octanoyl-rac-glycerol, 1-decanoyl-rac-glycerol, glycerol monolaurate (GML), rac-glycerol 1-myrstate, glycerol monocaprate, glycerol monocaprylate, glycerol monomyristate, or reutericyclin, wherein the second active ingredient is present at a concentration of 10 μg/ml to 100 mg/ml in the composition. The compositions are useful as antimicrobial agents.

### CROSS-REFERENCE TO RELATED APPLICATION

This application claims priority to U.S. Provisional Application No. 63/708,337 that was filed on Oct. 17, 2024. The entire content of the application referenced above is hereby incorporated by reference herein.

### BACKGROUND

Staphylococcus aureus bacteria are common commensal bacteria in the nose and other epithelial surfaces of humans. An estimate of colonization rates are from 30-40% depending on the age of the individual and any underlying conditions. As many as 70% of humans may be transiently colonized. For nearly 80% of patients being treated for hospital-associated infections, the infecting S. aureus bacteria are the same as those in the anterior nares. S. aureus bacteria most often originate from mucosal surfaces but can also cause infections across skin barriers. The US Centers for Disease Control (CDC) classifies S. aureus strains as USA 100 to 1100 base based on pulsed-field gel electrophoresis. Staphylococcus aureus is a highly significant infection problem in health care centers, particularly after surgery. Currently there is an on-going need for agents that are useful for treating or preventing S. aureus bacterial infections.

### SUMMARY

In certain embodiments, provided herein is an antimicrobial composition comprising (or consisting of): (a) a first active ingredient, wherein in the first active ingredient is selected from the group consisting of petroleum jelly, EpiCeram® emulsion, and/or glycerin, and (b) a second active ingredient selected from the group consisting of 1-octanoyl-rac-glycerol, 1-decanoyl-rac-glycerol, glycerol monolaurate (GML), rac-glycerol 1-myristate, glycerol monocaprate, glycerol monocaprylate, glycerol monomyristate, or reutericyclin, wherein the second active ingredient is present at a concentration of 10 μg/ml to 100 mg/ml in the composition. In certain embodiments, the composition further comprises (or consists of) (c) a probiotic bacterial strain, wherein the bacterial strain is lactobacillus and/or lactococci.

In certain embodiments, provided herein is a method of treating or preventing a bacterial infection in an animal comprising administering to the animal an antimicrobial composition comprising (or consisting of): (a) a first active ingredient, wherein in the first active ingredient is selected from the group consisting of petroleum jelly, EpiCeram® emulsion, and/or glycerin, and (b) a second active ingredient selected from the group consisting of 1-octanoyl-rac-glycerol, 1-decanoyl-rac-glycerol, glycerol monolaurate (GML), rac-glycerol 1-myrstate, glycerol monocaprate, glycerol monocaprylate, glycerol monomyristate, or reutericyclin, wherein the second active ingredient is present at a concentration of 10 μg/ml to 100 mg/ml in the composition. In certain embodiments, the composition further comprises (or consists of) (c) a probiotic bacterial strain, wherein the bacterial strain is lactobacillus and/or lactococci.

In certain embodiments, provided herein is a method for decolonizing bacteria on skin and/or in anterior nares in an animal comprising administering an antimicrobial composition comprising (or consisting of): (a) a first active ingredient, wherein in the first active ingredient is selected from the group consisting of petroleum jelly, EpiCeram® emulsion, and/or glycerin, and (b) a second active ingredient selected from the group consisting of 1-octanoyl-rac-glycerol, 1-decanoyl-rac-glycerol, glycerol monolaurate (GML), rac-glycerol 1-myrstate, glycerol monocaprate, glycerol monocaprylate, glycerol monomyristate, or reutericyclin, wherein the second active ingredient is present at a concentration of 10 μg/ml to 100 mg/ml in the composition. In certain embodiments, the composition further comprises (or consists of) (c) a probiotic bacterial strain, wherein the bacterial strain is lactobacillus and/or lactococci.

In certain embodiments, provided herein is a method of treating or preventing a bacterial infection in an animal comprising administering to the animal an antimicrobial composition comprising (or consisting of): (a) a first active ingredient, wherein in the first active ingredient is selected from the group consisting of petroleum jelly, EpiCeram® emulsion, and/or glycerin, and (b) a second active ingredient selected from the group consisting of 1-octanoyl-rac-glycerol, 1-decanoyl-rac-glycerol, glycerol monolaurate (GML), rac-glycerol 1-myrstate, glycerol monocaprate, glycerol monocaprylate, glycerol monomyristate, or reutericyclin, wherein the agent is present at a concentration of 10 μg/ml to 100 mg/ml in the composition, and administering a bacterial composition, wherein the bacterial composition comprises a bacterial strain and a pharmaceutically acceptable carrier.

In certain embodiments, provided herein is a method of killing S. aureus on a skin surface comprising administering the antimicrobial composition comprising (or consisting of): (a) a first active ingredient, wherein in the first active ingredient is selected from the group consisting of petroleum jelly, EpiCeram® emulsion, and/or glycerin, and (b) a second active ingredient selected from the group consisting of 1-octanoyl-rac-glycerol, 1-decanoyl-rac-glycerol, glycerol monolaurate (GML), rac-glycerol 1-myrstate, glycerol monocaprate, glycerol monocaprylate, glycerol monomyristate, or reutericyclin, wherein the second active ingredient is present at a concentration of 10 μg/ml to 100 mg/ml in the composition.

In certain embodiments, provided herein is a use of (a) a first active ingredient, wherein in the first active ingredient is selected from the group consisting of petroleum jelly, EpiCeram® emulsion, and/or glycerin, and (b) a second active ingredient selected from the group consisting of 1-octanoyl-rac-glycerol, 1-decanoyl-rac-glycerol, glycerol monolaurate (GML), rac-glycerol 1-myrstate, glycerol monocaprate, glycerol monocaprylate, glycerol monomyristate, or reutericyclin, to prepare a medicament for treating or preventing a skin infection in an animal.

In certain embodiments, provided herein is a method of treating or preventing a bacterial infection in an animal comprising co-administering to the animal an antimicrobial composition comprising (or consisting of): (a) a first active ingredient, wherein in the first active ingredient is selected from the group consisting of petroleum jelly, EpiCeram® emulsion, and/or glycerin, and (b) a second active ingredient selected from the group consisting of 1-octanoyl-rac-glycerol, 1-decanoyl-rac-glycerol, glycerol monolaurate (GML), rac-glycerol 1-myrstate, glycerol monocaprate, glycerol monocaprylate, glycerol monomyristate, or reutericyclin, wherein the second active ingredient is present at a concentration of 10 μg/ml to 100 mg/ml in the composition, and a composition comprising at least one bacterial strain in a pharmaceutically acceptable carrier, wherein the bacterial strain is lactobacillus and/or lactococci.

### BRIEF DESCRIPTION OF DRAWINGS

FIG. 1. Killing of toxic shock syndrome Staphylococcus aureus strain MN8 by GML alone (right bars) and GML+petrolatum (left bars) over 24 hours. S. aureus MN8 was added to GML in phosphate-buffered saline (PBS; 0.005M NaPO₄, 0.15M NaCl) or GML in petrolatum. Both the PBS and petrolatum were warmed to 40° C. to solubilize the GML (for petrolatum) and to add the S. aureus.The tubes were then incubated in triplicate at 37° C. without shaking for the 24-hour test period.

FIG. 2. Killing of necrotizing pneumonia, methicillin-resistant Staphylococcus aureus (MRSA) strain MNKN by GML alone (right bars) and GML+petrolatum (left bars) over 24 hours. S. aureus MNKN was added to GML in phosphate-buffered saline (PBS; 0.005M NaPO₄, 0.15M NaCl) or GML in petrolatum. Both the PBS and petrolatum were warmed to 40 ° C. to solubilize the GML (for petrolatum) and to add the S. aureus.The tubes were then incubated in triplicate at 37 ° C. without shaking for the 24-hour test period.

### DETAILED DESCRIPTION

### Antimicrobial Compositions

In certain embodiments, provided herein is an antimicrobial composition comprising (or consisting of): (a) a first active ingredient, wherein in the first active ingredient is selected from the group consisting of petroleum jelly, EpiCeram® emulsion, and/or glycerin, and (b) a second active ingredient selected from the group consisting of 1-octanoyl-rac-glycerol, 1-decanoyl-rac-glycerol, glycerol monolaurate (GML), rac-glycerol 1-myrstate, glycerol monocaprate, glycerol monocaprylate, glycerol monomyristate, or reutericyclin, wherein the second active ingredient is present at a concentration of 10 μg/ml to 100 mg/ml in the composition. In certain embodiments, the composition further comprises (or consists of) (c) a probiotic bacterial strain, wherein the bacterial strain is lactobacillus and/or lactococci.

In certain embodiments, the second ingredient is 1-octanoyl-rac-glycerol, 1-decanoyl-rac-glycerol, glycerol monolaurate (GML), rac-glycerol 1-myrstate, or reutericyclin. In certain embodiments, the second active ingredient is Glycerol Monolaurate (GML). GML is a naturally occurring fatty acid molecule and antimicrobial agent. GML suppresses the growth and virulence of numerous gram positive and gram-negative bacteria, fungi, and enveloped viruses. GML is on the FDA's Generally Recognized as Safe list (GRAS) and is incorporated in various products such as deodorants, lotions, and cosmetics. It is also widely available as a homeopathic supplement and is extensively used as a food preservative and emulsifier. GML is an effective antimicrobial that has extensive commercial and therapeutic uses.

In certain embodiments, the second active ingredient is glycerol monocaprate, glycerol monocaprylate, or glycerol monomyristate. In certain embodiments, the caprate, caprylate, and myristate are located at any position on glycerol. In certain embodiments, the caprate, caprylate, and myristate are located at the 1 (same as 3) position or the 2 position.

In certain embodiments, the composition consists of: (a) a first active ingredient, wherein in the first active ingredient is selected from the group consisting of petroleum jelly, EpiCeram® emulsion, and/or glycerin, and (b) a second active ingredient selected from the group consisting of 1-octanoyl-rac-glycerol, 1-decanoyl-rac-glycerol, glycerol monolaurate (GML), rac-glycerol 1-myrstate, glycerol monocaprate, glycerol monocaprylate, glycerol monomyristate, reutericyclin, wherein the second active ingredient is present at a concentration of 10 μg/ml to 100 mg/ml in the composition.

In certain embodiments, the antimicrobial further comprising (c) a probiotic bacterial strain, wherein the bacterial strain is lactobacillus and/or lactococci.

In certain embodiments, the probiotic bacterial strain is Lactobacilli.

In certain embodiments, the lactobacilli are L. rhamnosus, L. acidophilus, L. reuteri, L. gasseri, and/or L. crispatus.

In certain embodiments, the probiotic bacterial strain is Lactococci.

In certain embodiments, the bacterial strain is Lactococcus lactis.

In certain embodiments, the probiotic bacterial strain is present at a concentration of 10⁷ to 10¹⁰ CFUs in the composition, such as at 10⁷, 10⁸, 10⁹, or 10¹⁰ CFUs.

In certain embodiments, the antimicrobial composition consists of: (a) a first active ingredient, wherein in the first active ingredient is selected from the group consisting of petroleum jelly, EpiCeram® emulsion, and/or glycerin, (b) a second active ingredient selected from the group consisting of 1-octanoyl-rac-glycerol, 1-decanoyl-rac-glycerol, glycerol monolaurate (GML), rac-glycerol 1-myrstate, glycerol monocaprate, glycerol monocaprylate, glycerol monomyristate, or reutericyclin, and (c) a bacterial strain, wherein the bacterial strain is lactobacillus and/or lactococci., wherein the second active ingredient is present at a concentration of 10 μg/ml to 100 mg/ml in the composition. In certain embodiments, the second active ingredient is present at a concentration of 10, 20, 30, 40, 50, 60, 70, 80, 90, 100 μg/ml, or 1, 10, 20, 30, 40, 50, 60, 70, 80, 90 or 100 mg/ml in the composition.

Useful dosages of the second active ingredient can be determined by comparing their in vitro activity, and in vivo activity in animal models. Methods for the extrapolation of effective dosages in mice, and other animals, to humans are known to the art; for example, see U.S. Pat. No. 4,938,949.

The amount of the antibacterial composition required for use in treatment will vary with the route of administration, the nature of the condition being treated and the age and condition of the patient and will be ultimately at the discretion of the attendant physician or clinician.

The desired dose of the antibacterial composition may conveniently be presented in a single dose, or as divided doses administered at appropriate intervals, for example, as two, three, four or more sub-doses per day. The sub-dose itself may be further divided, e.g., into a number of discrete loosely spaced administrations.

### Methods of Using Antimicrobial Compositions

### Treating or Preventing a Bacterial Infection

In certain embodiments, provided herein is a method of treating or preventing a bacterial infection in an animal comprising administering to the animal an antimicrobial composition comprising (or consisting of): (a) a first active ingredient, wherein in the first active ingredient is selected from the group consisting of petroleum jelly, EpiCeram® emulsion, and/or glycerin, and (b) a second active ingredient selected from the group consisting of 1-octanoyl-rac-glycerol, 1-decanoyl-rac-glycerol, glycerol monolaurate (GML), rac-glycerol 1-myrstate, glycerol monocaprate, glycerol monocaprylate, glycerol monomyristate, or reutericyclin, wherein the second active ingredient is present at a concentration of 10 μg/ml to 100 mg/ml in the composition. In certain embodiments, the composition further comprises (or consists of) (c) a probiotic bacterial strain, wherein the bacterial strain is lactobacillus and/or lactococci.

In certain embodiments, the second active ingredient is present at a concentration of 10, 20, 30, 40, 50, 60, 70, 80, 90, 100 μg/ml, or 1, 10, 20, 30, 40, 50, 60, 70, 80, 90 or 100 mg/ml in the composition.

In certain embodiments, the method comprises administering the antimicrobial composition and separately administering a probiotic bacterial strain.

In certain embodiments, the antimicrobial composition and the bacterial composition are administered separately or simultaneously.

In certain embodiments, the probiotic bacterial strain is present at a concentration of 10⁷ to 10¹⁰ CFUs in the composition, such as at 10⁷, 10⁸, 10⁹, or 10¹⁰ CFUs.

### Decolonizing Bacteria on Skin And/or in Anterior

In certain embodiments, provided herein is a method for decolonizing bacteria on skin and/or in anterior nares in an animal comprising administering an antimicrobial composition comprising (or consisting of): (a) a first active ingredient, wherein in the first active ingredient is selected from the group consisting of petroleum jelly, EpiCeram® emulsion, and/or glycerin, and (b) a second active ingredient selected from the group consisting of 1-octanoyl-rac-glycerol, 1-decanoyl-rac-glycerol, glycerol monolaurate (GML), rac-glycerol 1-myrstate, glycerol monocaprate, glycerol monocaprylate, glycerol monomyristate, or reutericyclin, wherein the second active ingredient is present at a concentration of 10 μg/ml to 100 mg/ml in the composition. In certain embodiments, the composition further comprises (or consists of) (c) a probiotic bacterial strain, wherein the bacterial strain is lactobacillus and/or lactococci.

In certain embodiments, the second active ingredient is present at a concentration of 10, 20, 30, 40, 50, 60, 70, 80, 90, 100 μg/ml, or 1, 10, 20, 30, 40, 50, 60, 70, 80, 90 or 100 mg/ml in the composition.

In certain embodiments, the probiotic bacterial strain is present at a concentration of 10⁷ to 10¹⁰ CFUs in the composition, such as at 10⁷, 10⁸, 10⁹, or 10¹⁰ CFUs.

In certain embodiments, the method is for decolonizing skin.

In certain embodiments, the method is for decolonizing anterior nares.

### Treating or Preventing Diabetic Ulcers

In certain embodiments, provided herein is a method of treating or preventing diabetic ulcers comprising administering the antimicrobial composition comprising (or consisting of): (a) a first active ingredient, wherein in the first active ingredient is selected from the group consisting of petroleum jelly, EpiCeram® emulsion, and/or glycerin, and (b) a second active ingredient selected from the group consisting of 1-octanoyl-rac-glycerol, 1-decanoyl-rac-glycerol, glycerol monolaurate (GML), rac-glycerol 1-myrstate, glycerol monocaprate, glycerol monocaprylate, glycerol monomyristate, or reutericyclin, wherein the second active ingredient is present at a concentration of 10 μg/ml to 100 mg/ml in the composition. In certain embodiments, the composition further comprises (or consists of) (c) a probiotic bacterial strain, wherein the bacterial strain is lactobacillus and/or lactococci.

In certain embodiments, the second active ingredient is present at a concentration of 10, 20, 30, 40, 50, 60, 70, 80, 90, 100 μg/ml, or 1, 10, 20, 30, 40, 50, 60, 70, 80, 90 or 100 mg/ml in the composition.

In certain embodiments, the probiotic bacterial strain is present at a concentration of 10⁷ to 10¹⁰ CFUs in the composition, such as at 10⁷, 10⁸, 10⁹, or 10¹⁰ CFUs.

Killing S. aureus on a Skin Surface

In certain embodiments, provided herein is a method of killing S. aureus on a skin surface comprising administering the antimicrobial composition comprising (or consisting of): (a) a first active ingredient, wherein in the first active ingredient is selected from the group consisting of petroleum jelly, EpiCeram® emulsion, and/or glycerin, and (b) a second active ingredient selected from the group consisting of 1-octanoyl-rac-glycerol, 1-decanoyl-rac-glycerol, glycerol monolaurate (GML), rac-glycerol 1-myrstate, glycerol monocaprate, glycerol monocaprylate, glycerol monomyristate, or reutericyclin, wherein the second active ingredient is present at a concentration of 10 μg/ml to 100 mg/ml in the composition.

In certain embodiments, provided herein is a use of (a) a first active ingredient, wherein in the first active ingredient is selected from the group consisting of petroleum jelly, EpiCeram®emulsion, and/or glycerin, and (b) a second active ingredient selected from the group consisting of 1-octanoyl-rac-glycerol, 1-decanoyl-rac-glycerol, glycerol monolaurate (GML), rac-glycerol 1-myrstate, glycerol monocaprate, glycerol monocaprylate, glycerol monomyristate, or reutericyclin, to prepare a medicament for treating or preventing a skin infection in an animal.

In certain embodiments, the second active ingredient is present at a concentration of 10, 20, 30, 40, 50, 60, 70, 80, 90, 100 μg/ml, or 1, 10, 20, 30, 40, 50, 60, 70, 80, 90 or 100 mg/ml in the composition.

### Use of Antibacterial Compositions

In certain embodiments, provided herein is a use of (a) a first active ingredient, wherein in the first active ingredient is selected from the group consisting of petroleum jelly, EpiCeram® emulsion, and/or glycerin, and (b) a second active ingredient selected from the group consisting of 1-octanoyl-rac-glycerol, 1-decanoyl-rac-glycerol, glycerol monolaurate (GML), rac-glycerol 1-myrstate, glycerol monocaprate, glycerol monocaprylate, glycerol monomyristate, or reutericyclin, to prepare a medicament for treating or preventing a skin infection in an animal.

In certain embodiments, the medicament further comprises at least one bacterial strain, wherein the bacterial strain is lactobacillus and/or lactococci.

### General Features of the Methods

In certain embodiments of the methods described above, the administration is topical.

In certain embodiments of the methods described above, the animal is a mammal. In certain embodiments, the mammal is a human.

In certain embodiments of the methods described above, the probiotic bacterial strain is Lactobacilli.

In certain embodiments of the methods described above, the lactobacilli are L. rhamnosus, L. acidophilus, L. reuteri, L. gasseri, and/or L. crispatus.

In certain embodiments of the methods described above, the probiotic bacterial strain is lactococci.

In certain embodiments of the methods described above, the bacterial strain is Lactococcus lactis.

In certain embodiments, the probiotic bacterial strain is present at a concentration of 10⁷ to 10¹⁰ CFUs in the composition, such as at 10⁷, 10⁸, 10⁹, or 10¹⁰ CFUs.

In certain embodiments of the methods described above, the second active ingredient is 1-octanoyl-rac-glycerol, 1-decanoyl-rac-glycerol, glycerol monolaurate (GML), rac-glycerol 1-myrstate, glycerol monocaprate, glycerol monocaprylate, glycerol monomyristate, or reutericyclin. In certain embodiments, the second active ingredient is GML.

In certain embodiments of the methods described above, the animal is infected with bacteria. In certain embodiments, the bacterial infection is a Gram-positive bacterial strain infection. In certain embodiments, the Gram-positive bacterial strain is selected from the group consisting of Actinomyces naeslundii, Actinomyces viscosus, Bacillus anthracis, Bacillus cereus, Bacillus subtilis, Clostridium difficile, Corynebacterium diphtheriae, Corynebacterium ulcerans, Enterococcus faecalis, Enterococcus faecium, Micrococcus luteus, Mycobacterium avium, Mycobacterium intracellulare, Mycobacterium leprae, Mycobacterium tuberculosis, Propionibacterium acnes, Staphylococcus aureus, Staphylococcus epidermidis, Staphylococcus haemolyticus, Staphylococcus hominis, Staphylococcus hyicus, Staphylococcus intermedius, Staphylococcus saccharolyticus, Staphylococcus saprophyticus, Streptococcus agalactiae, Streptococcus mutans, Streptococcus pneumoniae, Streptococcus pyogenes, Streptococcus salivarius and Streptococcus sanguis.

The terms “treat,” “treatment, ”or “treating” to the extent it relates to a disease or condition includes inhibiting the disease or condition, eliminating the disease or condition, and/or relieving one or more symptoms of the disease or condition. The terms “treat,” “treatment,” or “treating” also refer to both therapeutic treatment and/or prophylactic treatment or preventative measures, wherein the object is to prevent or slow down (lessen) an undesired physiological change or disorder, such as, for example, the development or spread of infection. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder. “Treat,” “treatment,” or “treating” can also mean prolonging survival as compared to expected survival if not receiving treatment. Those in need of treatment include those already with the disease or disorder as well as those prone to have the disease or disorder or those in which the disease or disorder is to be prevented. In one embodiment “treat,” “treatment,” or “treating” does not include preventing or prevention.

The phrase “therapeutically effective amount” or “effective amount” includes but is not limited to an amount of a compound of the that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “mammal” as used herein refers to humans, higher non-human primates, rodents, domestic, cows, horses, pigs, sheep, dogs and cats. In one embodiment, the mammal is a human. The term “patient” as used herein refers to any animal including mammals. In one embodiment, the patient is a mammalian patient. In one embodiment, the patient is a human patient.

### Methods of Manufacture of Antimicrobial Compositions

In certain embodiments, Petrolatum is used as a GMP product for human use as a first active ingredient. In certain embodiments, EpiCeram® emulsion, and/or glycerin are used as a first active ingredient. In certain embodiments, Glycerol monolaurate (GML) and other monoesters are produced from food-grade product by GMP recrystallization as a second active ingredient. The two active ingredients are mixed at the desired concentrations and heated to 40 ° C. to solubilize the GML or other monoester in the first active ingredient (e.g., petrolatum). The final antimicrobial composition is stored at room temperature.

### Methods of Administration

For topical administration, the present compositions may be applied in pure form.

In certain embodiments, the antimicrobial composition is applied by swab or directly by fingers to affected areas to form a thin-film.

The invention will now be illustrated by the following non-limiting Examples.

### EXAMPLE 1

Staphylococcus aureus is a highly significant pathogen, causing a myriad of skin, soft tissue, and systemic infections. Among these infections are skin infections, overt and non-overt, in the 33 million Americans with diabetes mellitus type 2 and 30 million Americans with atopic dermatitis. Koch's postulates were recently fulfilled to show that S. aureus is an important cause of diabetes mellitus type 2. Patients with diabetes mellitus type 2 may develop serious and life-threatening ulcerative lesions, most of which are infected with S. aureus.Additionally, it has been shown that the organisms drive development and persistence of atopic dermatitis as well as the atopic march which includes atopic dermatitis and food allergy.

Glycerol monolaurate (GML) is a fatty acid monoester that is generally recognized as safe (GRAS) as a food additive, as determined by the Food and Drug Administration (FDA). GML is broadly antimicrobial, particularly for killing Gram-positive bacteria such as S. aureus and all enveloped viruses.

For example, 54 strains of S. aureus, including methicillin-sensitive and methicillin-resistant (MRSA), were tested and all 54 were killed by GML. One drawback to the used of GML is that S. aureus produces a lipase that can degrade GML. However, despite this drawback, S. aureus strains can be killed if GML is present at concentrations greater than the solubility limit in aqueous solutions. The use of an aqueous solution, however, creates another drawback: how to make a uniform suspension of GML that is not in solution when used.

Without being bound by theory, the target for GML to kill S. aureus appears to be embedding in the plasma membrane and interfering with signaling through staphylococcal two-component systems. It then appears that there is a rapid but progressive build-up of GML into the plasma membrane. As the GML builds up, more GML is solubilized, and greater build-up occurs until the bacteria die in 1-2 hours.

Pilot human studies have shown that GML can be used to decolonize the anterior nares of S. aureus, and GML can be added to tampons to interfere with vaginal S. aureus and its exotoxin production. In these studies, there were no adverse events reported. While doing these experiments, it was noticed that GML can be added to a nonaqueous gel (K-Y Warming Gel) and maintain anti-staphylococcal activity. It is important to note, however, that K-Y Warming gel cannot be used on open skin, as would be present in atopic dermatitis and diabetes mellitus type 2 (particularly ulcers), because the compound is painful to administer (e.g., stings).

It has also been shown that GML solubilized in K-Y Warming Gel kills enveloped viruses and is safe for chronic use vaginally as demonstrated in rhesus macaques. The killing mechanism of GML for enveloped viruses appears to be insertion into the vial envelope and prevention of fusion with human cells.

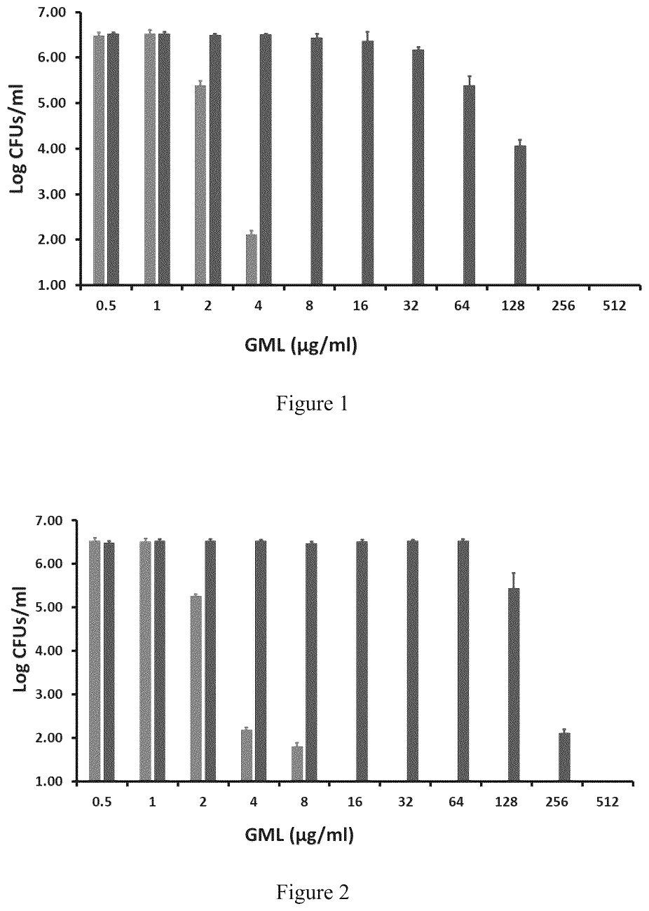

In certain embodiments provided herein are compositions of matter and uses for decolonization of human skin and anterior nares, wherein the composition comprises a combination of GML and petroleum jelly, with or without probiotic lactobacilli. As discussed further here, it has been recently shown that GML alone is not nearly as anti-staphylococcal as the combination of GML and petroleum jelly (see, FIGS. 1 and 2). Thus, the composition includes the combination of GML and petroleum jelly as one therapeutic, not GML alone. GML is highly soluble in petroleum jelly, with concentrations of GML greater than 1000,000 μg/ml solubility. It has been shown recently that probiotic lactobacilli are anti-staphylococcal.

Lactobacilli (L. rhamnosus, L. acidophilus, L. reuteri, L gasseri, and L. crispatus) and lactococci (Lactococcus lactis) are members of the normal microbiome of humans on both mucosal and skin surfaces. Thus, in certain embodiments, in addition to killing S. aureus on the skin and anterior nares of humans with diabetes mellitus type 2 and atopic dermatitis, these harmful bacteria are replaced with normal flora lactobacilli and lactococci when the S. aureus have been killed. This replacement of harmful bacteria with normal flora bacteria aids in preventing recurrences of S. aureus infections.

### EXAMPLE 2

Atopic dermatitis (AD) is the most common pruritic, chronic inflammatory skin condition, characterized by Type 2 immune responses, and other atopic co-morbidities including food allergy that are part of the atopic march. Every person with damaged skin will become infected with Staphylococcus aureus. These bacteria secrete multiple virulence factors that drive continuation of AD. This then leads to the chronicity of the condition. AD is initially treated with steroids, but millions of Americans develop a toxicity condition due to steroid use. There are also S. aureus strains that ramp up virulence factor production to overcome steroid treatment. In cases of patients who do not respond to steroids, biologics that neutralize Type 2 cytokines have become the therapy. This therapy is expensive must be continued indefinitely.

An alternative strategy has been developed. A Food and Drug Administration (FDA) generally recognized as safe (GRAS) compound glycerol monolaurate (GML) is a potent antimicrobial against S. aureus.Because there are more than 15 targets on S. aureus for GML, the bacteria are not able to develop resistance to GML. Additionally, GML is anti-inflammatory, including reducing Th2 immune responses. Thus, GML is useful in two ways to reduce AD. Further, GML is relatively inexpensive, with an estimated cost of 0.7 cents/treatment.

GML is related to a tetramic acid called reutericyclin, which is produced by a variety of lactobacilli strains, including some strains of L. crispatus.Women, who are colonized vaginally with L. crispatus producing reutericyclin, are mono-colonized with this organism through testing for at least five years. Both GML and reutericyclin are growth stimulants of lactobacilli which have an immunity gene against reutericyclin. No S. aureus have ever been identified that develop immunity to either GML or reutericyclin.

A composition is prepared that combines GML and L. crispatus, which produces reutericyclin, mixed in petroleum jelly for long-term function as novel therapeutics for AD. It is important to note that L. crispatus strains are typical probiotics for the gastrointestinal tract and vagina. Thus, replacement of the skin microbiome is unlikely to have deleterious effects.

Without being bound by theory, because both GML and reutericyclin are anti-inflammatory, it is hypothesized that treatment with GML and L. crispatus will result in down-regulation of the dysfunctional Type 2 response and reduce AD skin inflammation.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An antimicrobial composition comprising:
(a) a first active ingredient, wherein in the first active ingredient is selected from the group consisting of petroleum jelly, EpiCeram® emulsion, and/or glycerin, and
(b) a second active ingredient selected from the group consisting of 1-octanoyl-rac-glycerol, 1-decanoyl-rac-glycerol, glycerol monolaurate (GML), rac-glycerol 1-myrstate, glycerol monocaprate, glycerol monocaprylate, glycerol monomyristate, or reutericyclin, wherein the second active ingredient is present at a concentration of 10 μg/ml to 100 mg/ml in the composition.

2-5. (canceled)

6. The antimicrobial composition of claim 1, wherein the composition consists of:
(a) a first active ingredient consisting of petroleum jelly, and
(b) a second active ingredient consisting of glycerol monolaurate (GML),

7. The antimicrobial composition of claim 1, further comprising (c) a probiotic bacterial strain, wherein the bacterial strain is lactobacillus and/or lactococci.

8. The antimicrobial composition of claim 7, wherein the probiotic bacterial strain is Lactobacilli.

9. The antimicrobial composition of claim 8 wherein the lactobacilli are L. rhamnosus, L. acidophilus, L. reuteri, L. gasseri, and/or L. crispatus.

10. The antimicrobial composition of claim 7, wherein the probiotic bacterial is Lactococci.

11. The antimicrobial composition of claim 10, wherein the bacterial strain is Lactococcus lactis.

12. The antimicrobial composition of claim 7, wherein the probiotic bacterial strain is present at a concentration of 10⁷ to 10¹⁰ colony forming units (CFUs) in the composition.

13. (canceled)

14. A method of treating or preventing a bacterial infection, decolonizing bacteria on skin and/or in anterior nares, or treating or preventing diabetic ulcers in an animal comprising administering to the animal an antimicrobial composition of claim 1.

15-18. (canceled)

19. The method of claim 14, further comprising administering a bacterial composition, wherein the bacterial composition comprises a bacterial strain and a pharmaceutically acceptable carrier.

20. The method of claim 19, wherein the antimicrobial composition and the bacterial composition are administered separately or simultaneously.

21-22. (canceled)

23. The method of claim 14, wherein the method is for treating or preventing a bacterial infection, and the infection is a skin infection.

24. The method of claim 23, wherein the skin infection is an S. aureus infection on a skin surface.

25. The method of claim 23, further comprising colonizing the skin surface with at least one bacterial strain, wherein the bacterial strain is lactobacillus and/or lactococci.

26. The method of claim 25, wherein the bacterial strain is Lactobacilli.

27. The method of claim 26 wherein the lactobacilli are L. rhamnosus, L. acidophilus, L. reuteri, L. gasseri, and/or L. crispatus.

28. The method of claim 25, wherein the bacterial strain is Lactococci.

29. The method of claim 28, wherein the bacterial strain is Lactococcus lactis.

30. The method of claim 25, wherein the bacterial strain is present at a concentration of 10⁷ to 10¹⁰ in the petroleum jelly.

31-40. (canceled)

41. The method of claim 14, wherein the method is a method of treating or preventing a bacterial infection, decolonizing bacteria on skin and/or in anterior nares, and the bacterial infection is a Gram-positive bacterial strain infection.

42. The method of claim 41, wherein the Gram-positive bacterial strain is selected from the group consisting of Actinomyces naeslundii, Actinomyces viscosus, Bacillus anthracis, Bacillus cereus, Bacillus subtilis, Clostridium difficile, Corynebacterium diphtheriae, Corynebacterium ulcerans, Enterococcus faecalis, Enterococcus faecium, Micrococcus luteus, Mycobacterium avium, Mycobacterium intracellulare, Mycobacterium leprae, Mycobacterium tuberculosis, Propionibacterium acnes, Staphylococcus aureus, Staphylococcus epidermidis, Staphylococcus haemolyticus, Staphylococcus hominis, Staphylococcus hyicus, Staphylococcus intermedius, Staphylococcus saccharolyticus, Staphylococcus saprophyticus, Streptococcus agalactiae, Streptococcus mutans, Streptococcus pneumoniae, Streptococcus pyogenes, Streptococcus salivarius and Streptococcus sanguis.

43. The method of claim 14, wherein the administration is topical.
